(19) United States
(12) Patent Application Publication
(10) Pub. No.: US 2016/0038704 A1
(43) Pub. Date: Feb. 11, 2016

(54) MEDICAL TUBE HOLDER

(71) Applicant: XIGENT INNOVATIONS PTY LTD, Greenbank, Queensland (AU)

(72) Inventor: Duncan Murray, Greenbank, Queensland (AU)

(73) Assignee: XIGENT INNOVATIONS PTY LTD, Greenbank, Queensland (AU)

(21) Appl. No.: 14/781,321

(22) PCT Filed: Apr. 11, 2014
(86) PCT No.: PCT/AU2014/000389

§ 371 Date: Sep. 30, 2015

(30) Foreign Application Priority Data
Apr. 11, 2013 (AU) 2013203636

(51) Int. Cl. A61M 16/04

(52) U.S. Cl. CPC A61M 16/0497; A61M 2025/022

(57) ABSTRACT

A medical tube holder, for securing a tube relative to a patient, comprises an elongate patient engaging portion for passing around at least a part of a neck and mouth region of a patient; a tube holding portion, connected to the patient engaging portion, for passing around at least a part of a medical tube; a tube-holding-portion securing arrangement for securing the tube holding portion around at least a part of the medical tube so that the medical tube can be securely positioned relative to the patient; and a patient-engaging-portion securing arrangement for securing the patient engaging portion around the patient's neck and mouth region to secure the medical tube holder to the patient. The patient-engaging-portion securing arrangement has a tightening arrangement to facilitate tightening of the patient engaging portion around the patient's neck and mouth region, and a locking arrangement that prevents loosening of the patient engaging portion while allowing further tightening.

### TECHNICAL FIELD

The present disclosure relates to a medical tube holder. More particularly, but not exclusively, the present disclosure relates to an endotracheal tube holder to secure an endotracheal tube in an airway of a patient.

### BACKGROUND

Endotracheal intubation is the placement of an endotracheal tube into the trachea. It plays an important role in airway management, and is often vital in the care of patients with an altered level of consciousness, patients under or recovering from anesthesia, patients requiring resuscitation, and within the intensive care setting. Endotracheal intubation is performed in the prehospital as well as the hospital settings. Placement of an endotracheal lube requires training and practice, and the tube must be placed in the correct position. Subsequent movement of the tube may have life threatening or fatal consequences. If the tube is moved too far into the airway it may result in ventilation of only one lung with consequent problems with hypoxia and barotraumas. If the tube is moved too far out it may result in extubation, resulting in a loss of airway protection and an inability to ventilate the patient.

The endotracheal tube is generally attached to a ventilation device of some kind, and the patient may also need to be moved whilst intubated and ventilated, increasing the chance of the tube becoming moved or dislodged.

It is hence important to secure the endotracheal tube in position.

Devices other than endotracheal tubes (such as the Laryngeal Mask Airway) are also used in airway management. They also require a way of keeping them securely in place.

Many techniques have been used to secure these tubes in place: from twill tape (using various knots), adhesive tape, to various commercial devices. Twill tape is commonly used, and has the benefits of being cheap and readily available. It can, however, be difficult to pass under the patient's neck and tie securely, particularly in an emergency situation. Adhesive tape is also commonly used, but may fail to securely hold a tube relative to a patient due to lack of adhesion to sweaty or contaminated skin. Currently available commercial devices are often expensive and may be slow to apply, may be complex and may require users to undergo an undesirably large amount of training in their use in order to provide the desired levels of safety and effectiveness.

### SUMMARY OF THE DISCLOSURE

According to a first aspect of the present disclosure there is provided a medical tube holder, for securing a tube relative to a patient, comprising:

an elongate patient engaging portion for passing around at least a part of a neck and mouth region of a patient;

a tube holding portion, connected to the patient engaging portion, for passing around at least a part of a medical tube;

a tube-holding-portion securing arrangement for securing the tube holding portion around at least a part of the medical tube so that the medical tube can be securely positioned relative to the patient; and

a patient-engaging-portion securing arrangement for securing the patient engaging portion around the patient's neck and mouth region to secure the medical tube holder to the patient, the patient-engaging-portion securing arrangement comprising:
a patient-engaging-portion tightening arrangement to facilitate tightening of the patient engaging portion around the patient's neck and mouth region; and
a patient-engaging-portion locking arrangement that prevents loosening of the patient engaging portion while allowing further tightening of the patient engaging portion around the neck and mouth region of the patient.

In an embodiment the patient-engaging-portion locking arrangement comprises a toothed ratchet portion and a pawl configured to engage the toothed ratchet portion.

In an embodiment the toothed ratchet portion is provided at a first end region of the elongate patient engaging portion and the pawl is provided at a second end region of the elongate patient engaging portion.

In an embodiment the patient-engaging-portion tightening arrangement comprises an aperture defined by a body provided at the first end region of the elongate patient engaging portion through which at least part of the second end region of the elongate patient engaging portion may be pulled.

In an embodiment the pawl of the patient-engaging-portion locking arrangement is attached to the body.

In an embodiment the patient-engaging-portion locking arrangement allows unlocking of the patient engaging portion.

In an embodiment the pawl is adapted to be operated to at least partially disengage the toothed ratchet portion in order to allow unlocking.

In an embodiment the tube-holding-portion securing arrangement comprises: a tube-holding-portion tightening arrangement to facilitate tightening of the tube holding portion around the tube; and

a tube-holding-portion locking arrangement that prevents loosening of the tube holding portion while allowing further tightening of the tube holding portion around the tube.

In an embodiment the patient engaging portion comprises a wider central region and at least one narrower end region, and the tube holding portion is provided at or adjacent the at least one narrower end region.

In use, this can allow the at least one narrower end region to be positioned across the patient's mouth, the narrower width facilitating oral access by medical staff for oral suctioning or other procedures (such as insertion of an orogastric tube). The wider central region can assist in decreasing issues with pressure areas on the patient's skin.

In an embodiment the patient-engaging-portion securing arrangement is integrally formed with the patient engaging portion.

In an embodiment the tube-holding-portion securing arrangement is integrally formed with the tube holding portion.

In an embodiment the tube-holding-portion securing arrangement is integrally formed with the patient engaging portion.

In an embodiment the entire medical tube holder is integrally formed from a single piece of plastic, for example by injection molding.

In an embodiment, upon activation of the patient-engaging-portion securing arrangement at least a part of the patient engaging portion defines a first loop substantially in a first plane and upon activation of the tube-holding-portion securing arrangement, at least a part of the tube holding portion at least partially defines a second loop substantially in a second plane. The first plane and the second plane may be mutually perpendicular. This can assist correct orientation of the medical tube relative to the patient.

In an embodiment upon activation of the tube-holding-portion securing arrangement at least part of the tube holding portion partially defines a loop adapted to surround a medical tube, and said loop is partially defined by part of the patient engaging portion.

In an embodiment the tube holding portion comprises an elongate flexible member one end of which is permanently attached to the patient engaging portion and a portion of the elongate flexible member spaced apart from said one end is fastenable to the patient engaging portion by the tube-holding-portion securing arrangement.

In an embodiment the tube holding portion is arranged relative to the patient-engaging-portion securing arrangement so that tube holding portion can be appropriately positioned for securing a medical tube and retaining the medical tube in the mouth and an airway of the patient by positioning the patient-engaging-portion securing arrangement adjacent one side of the mouth of the patient.

According to a second aspect of the present disclosure there is provided a medical tube holder, for securing a tube relative to a patient, comprising:

an elongate patient engaging portion for passing around at least a part of a neck and mouth region of a patient to secure the medical tube holder to the patient; and

a tube holding portion for passing around at least a part of a medical tube to secure the medical tube relative to the medical tube holder;

wherein the patient engaging portion and the tube holding portion are configured to retain and support the tube in position with reference to the mouth of the patient; and wherein the patient engaging portion is integrally formed with the tube holding portion.

In an embodiment the medical tube holder further comprises:

a patient-engaging-portion securing arrangement for securing the patient engaging portion around at least a part of a neck and mouth region of a patient; and

a tube-holding-portion securing arrangement adapted for securing the tube to the tube holding portion and positioning the tube in the mouth of the patient;

wherein the patient-engaging-portion securing arrangement and the tube-holding-portion securing arrangement are adapted to retain and support the tube in position relative to the mouth of the patient.

In an embodiment the patient-engaging-portion securing arrangement further comprises:

a patient-engaging-portion tightening arrangement to facilitate tightening of the patient engaging portion around the patient's neck and mouth region; and

a patient-engaging-portion locking arrangement that prevents loosening of the patient engaging portion while allowing further tightening of the patient engaging portion around the neck and mouth region of the patient.

In an embodiment the tube-holding-portion securing arrangement further comprises:

a tube-holding-portion tightening arrangement to facilitate tightening of the tube holding portion around the tube; and

a tube-holding-portion locking arrangement that prevents loosening of the tube holding portion while allowing further tightening of the tube holding portion around the tube.

In an embodiment the patient-engaging-portion securing arrangement and/or the tube-holding-portion securing arrangement further comprise at least one toothed ratchet portion, a pawl configured to engage said at least one toothed ratchet and a through hole for receiving the toothed ratchet portion therein.

In an embodiment the patient-engaging-portion securing arrangement is integrally formed with the patient engaging portion and wherein the tube-holding-portion securing arrangement is integrally formed with the tube holding portion.

In an embodiment the tube-holding-portion securing arrangement is integrally formed with the patient engaging portion.

In an embodiment the entire medical tube holder is integrally formed from a single piece of plastic, for example by injection molding.

In an embodiment upon activation of the tube-holding-portion securing arrangement at least part of the tube holding portion partially defines a loop adapted to surround a medical tube, and wherein said loop is partially defined by part of the patient engaging portion.

In an embodiment the tube holding portion comprises an elongate flexible member, one end of which is permanently attached to the patient engaging portion and a portion of the elongate flexible member spaced apart from said one end is fastenable to the patient engaging portion by the tube-holding-portion securing arrangement.

In an embodiment the tube holding portion is arranged relative to the patient-engaging-portion securing arrangement so that tube holding portion can be appropriately positioned for securing a medical tube and retaining the medical tube in the mouth and an airway of the patient by positioning the patient-engaging-portion securing arrangement adjacent one side of the mouth of the patient.

According to a third aspect of the present disclosure there is provided a medical tube holder, for securing a tube relative to a patient, comprising:

an elongate patient engaging portion that defines at least a part of a first loop for passing around at least a part of a patient's neck and mouth region;

a tube holding portion, connected to the patient engaging portion, defining a first part of a second loop for passing around at least a part of the tube, wherein a second part of the second loop is defined by part of the patient engaging portion;

wherein the patient engaging portion and the tube holding portion are adapted to retain and support the tube in position relative to the mouth of the patient.

In an embodiment the medical tube holder further comprises:

a patient-engaging-portion securing arrangement for securing the patient engaging portion around the neck and mouth region of a patient; and

a tube-holding-portion securing arrangement for securing the tube to the tube holding portion and positioning the tube in the mouth of the patient;

wherein the patient-engaging-portion securing arrangement and the tube-holding-portion securing arrangement are adapted to retain and support the tube in position with reference to the airway of the patient.

In an embodiment the patient-engaging-portion securing arrangement further comprises:

a patient-engaging-portion tightening arrangement to facilitate tightening of the patient engaging portion around the patient's neck and mouth region; and

a patient-engaging-portion locking arrangement that prevents loosening of the patient engaging portion while allowing further tightening of the patient engaging portion around the neck and mouth region of the patient.

In an embodiment the tube-holding-portion securing arrangement further comprises:

a tube-holding-portion tightening arrangement to facilitate tightening of the tube holding portion around the tube; and

a tube-holding-portion locking arrangement that prevents loosening of the tube holding portion while allowing further tightening of the tube holding portion around the tube.

In an embodiment the patient-engaging-portion securing arrangement and/or the tube-holding-portion securing arrangement further comprise at least one toothed ratchet portion, a pawl configured to engage said at least one toothed portion; and a through hole for receiving the toothed portion therein.

In an embodiment upon activation of the patient-engaging-portion securing arrangement at least a part of the patient engaging portion defines a first loop substantially in a first plane and wherein upon activation of the tube-holding-portion securing arrangement, at least a part of the tube holding portion at least partially defines a second loop substantially in a second plane, and wherein the first plane and the second plane are mutually perpendicular.

In an embodiment upon activation of the tube-holding-portion securing arrangement at least part of the tube holding portion partially defines a loop adapted to surround a medical tube, and wherein said loop is partially defined by part of the patient engaging portion.

In an embodiment the tube holding portion comprises an elongate flexible member one end of which is permanently attached to the patient engaging portion and wherein a portion of the elongate flexible member spaced apart from said one end is fastenable to the patient engaging portion by the tube-holding-portion securing arrangement.

In an embodiment the tube holding portion is arranged relative to the patient-engaging-portion securing arrangement so that tube holding portion can be appropriately positioned for securing a medical tube and retaining the medical tube in the mouth and an airway of the patient by positioning the patient-engaging-portion securing arrangement adjacent one side of the mouth of the patient.

In an embodiment the entire medical tube holder is integrally formed from a single piece of plastic.

According to a fourth aspect of the present disclosure there is provided a medical tube holder for securing a tube to a patient, the apparatus comprising:

an elongate patient engaging portion for passing around at least a part of a patient's neck and mouth region;

a patient-engaging-portion securing arrangement for securing the patient engaging portion around at least a part of a patient's neck and mouth region;

a tube holding portion comprising an elongate flexible member, for passing around at least a part of the tube, one end of which is permanently attached to the patient engaging portion at an attachment region;

a tube-holding-portion securing arrangement for securing the tube in the airway of the patient, the tube-holding-portion securing arrangement adapted to secure part of the tube holding portion other than said one end to the patient engaging portion and/or the attachment thereby securing and retaining the tube relative to a mouth of the patient.

In an embodiment the patient-engaging-portion securing arrangement further comprises

a patient-engaging-portion tightening arrangement to facilitate tightening of the patient engaging portion around the patient's neck and mouth region; and

a patient-engaging-portion locking arrangement that prevents loosening of the patient engaging portion while allowing further tightening of the patient engaging portion around the neck and mouth region of the patient.

In an embodiment the tube-holding-portion securing arrangement further comprises:

a tube-holding-portion tightening arrangement to facilitate tightening of the tube holding portion around the tube; and

a tube-holding-portion locking arrangement that prevents loosening of the tube holding portion while allowing further tightening of the tube holding portion around the tube.

In an embodiment the patient-engaging-portion securing arrangement and/or the tube-holding-portion securing arrangement further comprise at least one toothed ratchet portion, a pawl configured to engage said at least one toothed portion; and a through hole for receiving the toothed portion therein.

According to a fifth aspect of the present disclosure there is provided a medical tube holder, for securing a tube relative to a patient, comprising:

an elongate patient engaging portion for passing around at least a part of a neck and mouth region of a patient;

a tube holding portion, connected to the patient engaging portion, for passing around at least a part of a medical tube;

wherein the elongate patient engaging portion includes a positioning indicator thereon, and wherein the tube holding portion is arranged relative to the positioning indicator so that when the positioning indicator is positioned adjacent one side of the mouth of a patient the tube holding portion is appropriately positioned for securing a medical tube and retaining the medical tube in the mouth and an airway of the patient.

In an embodiment the medical tube holder further comprises a patient-engaging-portion securing arrangement for securing the patient engaging portion around the patient's neck and mouth region to secure the medical tube holder to the patient; and the patient-engaging-portion securing arrangement acts as the positioning indicator.

In an embodiment the tube holding portion is arranged relative to the patient-engaging-portion securing arrangement so that tube holding can be appropriately positioned for securing a medical tube and retaining the medical tube in the mouth and an airway of the patient by positioning the patient-engaging-portion securing arrangement adjacent one side of the mouth of the patient.

In an embodiment the entire medical tube holder is integrally formed from a single piece of plastic.

It will be appreciated that a medical tube holder in accordance with any one of the above aspects may also include features described in relation to one or more of the other aspects, or embodiments thereof

Also described is a method of securing a medical tube relative to a patient, the method comprising the steps of:

passing an elongate patient engaging portion of a holder as described above, around at least a part of a neck and mouth region of a patient;

passing a tube holding portion of the holder around at least a part of a medical tube;

securing the medical tube relative to the mouth of the patient.

### BRIEF DESCRIPTION OF THE DRAWINGS

Notwithstanding any other forms which may fall within the scope of the apparatus and method as set forth in the Summary, specific embodiments will now be described, by way of example only, with reference to the accompanying drawings in which:

FIG. 1 is a view of an embodiment of a tube holder from above and one side;

FIG. 2A is an enlarged plan view of a first end of the tube holder of FIG. 1;

FIG. 2B is a view generally corresponding to the view of FIG. 2A, but more from one side;

FIG. 3 is an enlarged perspective view of the first end of the tube holder of FIGS. 1 to 2B;

FIG. 4 is a cross sectional view on IV-IV of FIG. 2A;

FIG. 5 is a cross sectional view on V-V of FIG. 2A;

FIG. 6 is a plan view of the embodiment of FIGS. 1 to 5 schematically illustrating a tube holding portion of the tube holder in operation, holding a tube;

FIG. 7 is an illustration of a first stage of operating a tube holder to secure a tube to a simulated patient;

FIG. 8 is an illustration of a second stage of operating a tube holder to secure a tube to a simulated patient; and

FIG. 9 is an illustration of a tube holder securing a tube to a simulated patient with the securing operation completed.

### DETAILED DESCRIPTION OF SPECIFIC EMBODIMENTS

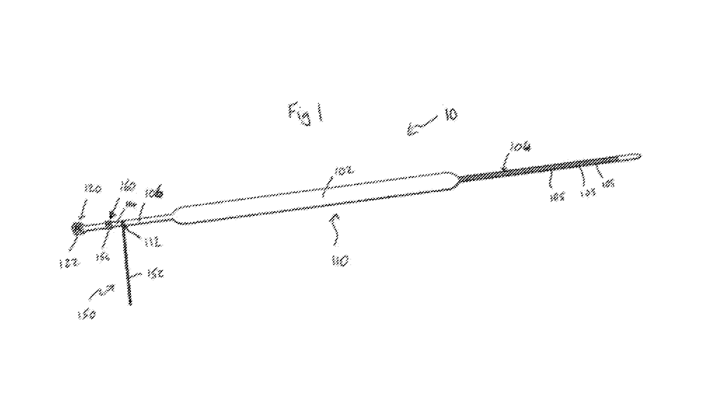

Referring to FIGS. 1 to 3, an embodiment of a medical tube holder in the form of a tube holder 10 is shown. The tube holder 10 includes two portions, namely a patient engaging portion in the form of a neck and head engaging portion 110 and a tube holding portion in the form of a tube holder portion 150.

The neck engaging portion 110 includes a centrally located neck band section 102 located in between terminating end sections 104 and 106 located at two ends of the neck engaging portion 110. The neck band section 102 is broader than the end sections 104 and 106. When used to secure a tube relative to a patient the neck engaging portion 110 may be passed under the patient's neck from a lateral side of the patient to position the neck band section 102 for placement around the patient's head and neck/mouth region thereby forming a loop around the patient's head and neck region. A patient-engaging-portion securing arrangement in the form of a first securing arrangement 120 is provided for securing the neck engaging portion 110 around a patient's neck and mouth region. The first securing arrangement 120, may be in the form of a ratchet and pawl arrangement as will he described in due course, and may be used for securing the neck and head engaging portion 110 to form a suitably tight first loop around the patient's head and neck/mouth region. The first securing arrangement 120 effectively secures the end sections 104 and 106 to each other by way of fastener 122. In the illustrated embodiment, the fastener 122 is integrally formed with the end section 106 and is located at the free end of end section 106.

The tube holder portion 150 of the tube holder 10 includes a tube holder body 152 that is of an elongate configuration in the illustrated embodiment. The tube holder body 152 is connected at one end to the neck engaging portion 110, at connection 112. The tube holder body 152 is connected at its first end by being integrally formed with a part of the neck engaging portion 110. In the illustrated embodiment, the tube holder body 152 is connected by way of being integrally formed with the end section 106 of the neck engaging portion 110. A tube holding portion securing arrangement in the form of a second securing arrangement 160 is provided for securing a tube T to the tube holder body 152 during use. The second securing arrangement 160 includes fastener 154 for fastening a part of the tube holder body 152 which is spaced from the connection 112 to the neck engaging portion 110, in use. During use, the tube holder body 152 passes at least part of the way around an external wall of a tube (T) and the second securing arrangement 160 secures the tube (T) by fastening the tube holder body 152 at some point along its length, as shown in FIG. 6 and as will be appreciated from the overall description. Therefore, the elongate tube holder body 152 forms at least part of a loop, provided by the tube holder 10 around the tube (T). The second securing arrangement 160 helps secure the tube T by fastening the tube holder body 152 to the neck engaging portion 110 providing the loop which entraps, and can securely hold, the tube T.

Having the tube holder body 152 connect, in use, at two distinct positions on the neck engaging portion 110, allows the plane of a loop defined by the tube holder body 152 (relative to the neck engaging portion 110) to be defined. (This is not least because the tube holder body is relatively transversely flexible in the plane of the desired loop, and relatively stiff in its other transverse directions, as is illustrated by the dimensions of the tube holder body 152, as shown in FIG. 3.) In use, the tube holder body 152 will tend to retain the tube T with the axis of the tube T perpendicular to the plane of the loop. This can allow the tube holder 10 to support a tube in a predetermined orientation relative to a patient in use, which can be of considerable assistance in securing a medical tube in a desired position relative to the patient's mouth and airways. However, the present disclosure is not limited to connecting the tube holder body 152 at two distinct positions on the neck engaging portion 110. In an alternative embodiment, the tube holder body may be connected to the neck engaging portion 110 at a single position, for example by having the fastener 154 positioned at the point where the tube holder body 152 is connected permanently to the neck engaging portion 110 or by way of providing an intermediate connecting member that connects the tube holder body 152 with the neck engaging portion 110 and providing the fastener at that intermediate connecting member.

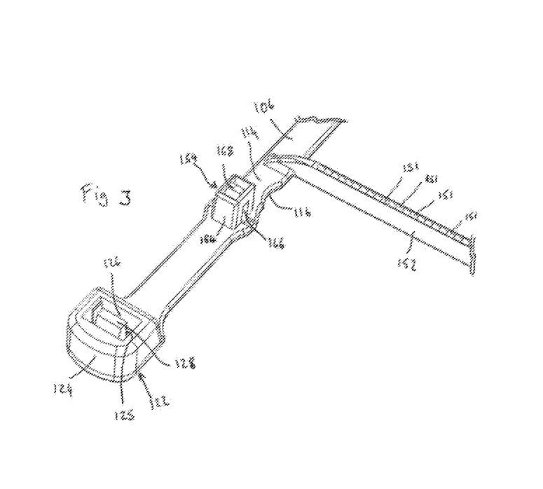

Referring to FIG. 4, the first securing arrangement 120 includes the fastener 122 for securing the neck engaging portion 110 to the patient's neck and mouth region. The fastener 122 has a fastener body 124 preferably but not necessarily formed of a single piece of preferably plastic material, such as nylon. The fastener 122 has an opening 126 for receiving an end section 104 of the neck engaging portion 110. The opening 126 is preferably of a size and shape to accommodate at least a part of end section 104 of the neck engaging portion 110. The fastener 122 includes pawl 128, slightly movable relative to the fastener body 124, that has engaging pawl portions 125 for engaging ridges in the form of ratchet teeth 105 located on the end section 104 of the neck engaging portion 110. During a securing operation, end section 104 of the neck engaging portion 110 is received in the opening 126. The ratchet teeth 105 are provided on an in-use outer surface of the neck engaging portion 110 (the top surface of section 104 as illustrated in FIG. 1). The engagement of the ratchet teeth 105 with the engaging pawl portions 125 enables the fastener 122 to securely retain end section 104. Furthermore, provision of the ratchet teeth 105 on the neck engaging portion 110, and the pawl 128 with engaging pawl portions 125, also provides a tightening mechanism. During use of the tube holder 10 end section 104 is manually passed into the opening 126. As the end section 104 is passed through the opening 126, neck engaging portion 110 which in use passes around the patient's head and neck region, progressively gets tensioned against the patient's head and neck region. Advantageously, providing ratchet teeth 105 on the neck engaging portion 110 and provision of engaging pawl portions 125 also provides a locking mechanism to prevent a decrease in tensioning of the neck engaging portion 110 during use. During use, the ratchet teeth 105 on the neck engaging portion 110 and the engaging pawl portions 125 form a complementary interlocking configuration that facilitates further tightening if desired, but. substantially prevents any withdrawal of the end section 104 of the neck engaging portion 110 from the opening 126, even when substantial force is applied in the withdrawal direction. As a result, the locking mechanism prevents a decrease in tensioning, or loosening, of the neck engaging portion 110 without preventing the further tightening or increase in tension of the neck engaging portion 110.

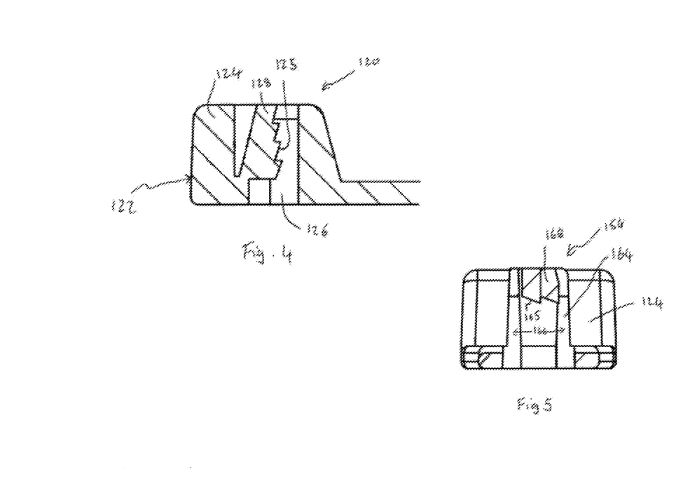

Referring to FIG. 5, the second securing arrangement 160 includes the fastener 154 for securing tube T during use to the tube holder portion 150. The fastener 154 has a fastener body 164 preferably but not necessarily formed of a single piece of preferably plastic material. The fastener 154 has an opening 166 for receiving a section of the tube holder body 152. The opening 166 is preferably of a size and shape to accommodate at least a part of the tube holder body 152. The fastener 154 includes pawl 168, slightly movable relative to the fastener body 164, that has engaging pawl portions 165 for engaging ridges, or ratchet teeth 151, located on a surface of the tube holder body 152 (the top surface of the tube holder body 152 as illustrated in FIGS. 1 to 3). During a securing operation, a section of the tube holder body 152 is received in the opening 166. The engagement of the ratchet teeth 151 located on the tube holder body 152 with the engaging pawl portions 165 enables the fastener 154 to fasten with the tube holder body 152 to form a tube engaging loop, as shown in FIG. 6. Furthermore, provision of the ratchet teeth 151 on the tube holder body 152 and provision of the engaging pawl portions 165 also provides a tightening mechanism. During use of the tube holder 10 a part of the tube holder body 152 is passed through the opening 166. As the tube holder body 152 is passed through the opening 166, the tube holder body 152 also passes around the tube T (see FIG. 6) and the tube holder body 152 progressively gets tensioned against outer walls of the tube T. Advantageously, providing ratchet teeth 151 on the tube holder body 152 and provision of engaging pawl portions 165 also provides a locking mechanism to prevent a decrease in tensioning of the tube holder body 152 during use. During use, the ratchet teeth 151 on the tube holder body 152 and the engaging pawl portions 165 form a complementary and interlocking configuration that facilitates further tightening if desired, but substantially prevents any withdrawal of the tube holder body 152 from the opening 166 due to the interlocking of the ratchet teeth 151 and the engaging pawl portions 165, even when substantial force is applied in the withdrawal direction. As a result, the locking mechanism prevents a decrease in tensioning, or loosening, of the tube holder portion without preventing continued or subsequent tightening, or increase in tensioning, of the tube holder body 152.

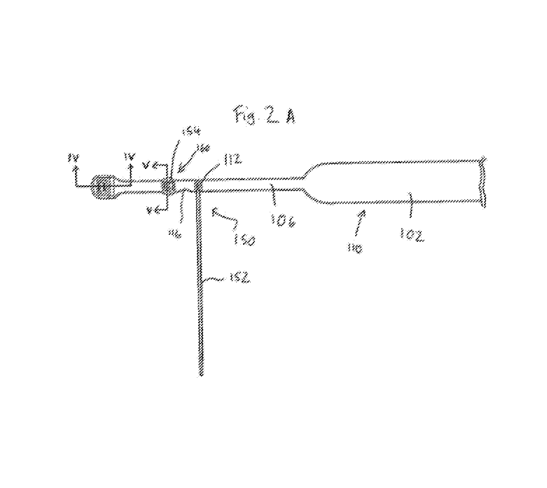

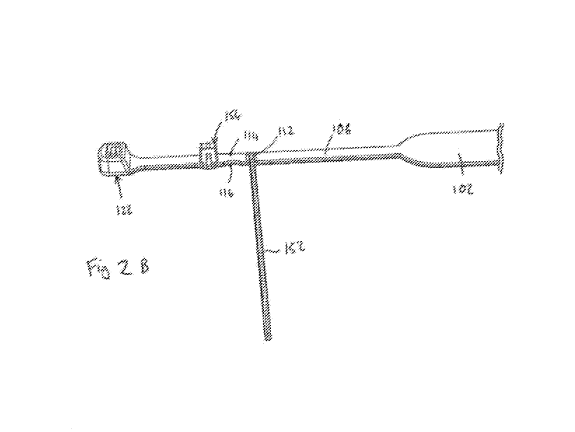

As shown in FIG. 6, in this embodiment the elongate tube holder body 152 forms part of the loop which surrounds and secures the tube T, and some of the loop is provided by a region of the neck engaging portion 110. More specifically, in this embodiment, some of the loop is provided by a part 114 of the neck engaging portion 110 between the connection 112 and the fastener 154. In this embodiment the neck engaging portion 110 is provided with a convex (or cut-out or contoured) region 116, for receiving an external surface of the tube (as also shown in FIGS. 2A, 2B and 3).

It will be appreciated that the mechanisms used to allow tightening of the engaging portion 110 and the tube holder body 152, may be identical or similar in construction and function to the mechanisms used in zip ties (also referred to as cable ties) which are known per se. Zip ties have a substantial history of use and commercially available embodiments have been found to be easy to manually tighten but extremely secure against inadvertent loosening, and to be extremely robust in use. Consequently, it will be appreciated that the described embodiments of tube holder are easy to manually tighten but extremely secure against inadvertent loosening, and are robust in use. Embodiments are preferably injection moulded from a suitable plastic. Nylon, for example, has been found to be suitable. The described embodiments are intended to be used once, and to be cut off a patient and discarded when removal from the patient is desired. The described embodiments can thus provide a medical tube holder which is easy and quick to apply, extremely secure, and robust. They are also economical to mass produce. The described embodiments may have particular value in emergency settings. It is possible that some embodiments may provide lesser patient comfort than some known methods of securing a medical tube, such as an endotracheal medical tube, to a patient, but the benefits (for example potentially life-saving benefits) are considered to considerably outweigh this potential disadvantage, especially in emergency settings.

It should be appreciated that the mechanisms used to allow tightening of the engaging portion 110 and the tube holder body 152, and described in relation to the illustrated embodiment as including features identical or similar in construction and function to the mechanisms used in zip ties may be varied as desired. In particular features or functions of zip ties which are known per se may be incorporated into embodiments of tube holders if desired. For example, releasable zip ties are known, and if desired if desired one or more of the fastening mechanisms of a tube holder in accordance with the present disclosure may be releasable analogously to a releasable zip tie. For example one or more of the fastening mechanisms may be constructed in a manner which allows its pawl mechanism to be temporarily moved by a user (eg by a finger or thumb of a user or by a suitably commonly available medical tool) to allow release of the teeth and consequently unlocking of the mechanism to allow at least partial loosening. This may provide additional functionality, for example to loosen a neck engaging portion 110 which has inadvertently been over tightened, rather than cutting it off the patient and using a new tube holder.

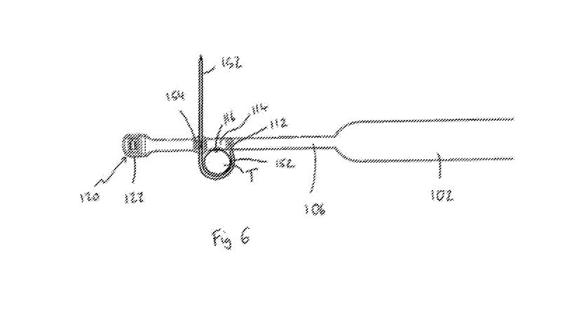

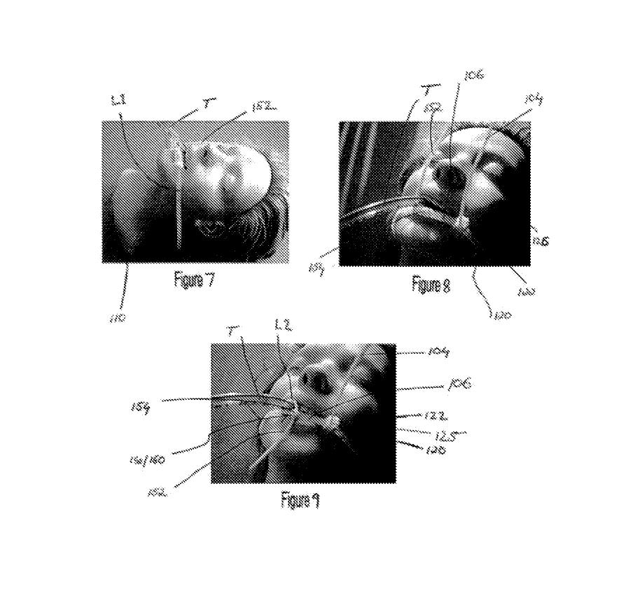

Referring to FIGS. 7 to 9 use of a tube holder 10 is illustrated. Neck engaging portion 110 is passed under the patient's neck from a lateral left side of the patient (the patient's right side) to position the neck band section 102 (not shown in FIGS. 7 to 9) around the patient's head and neck/mouth region. The first securing arrangement 120 secures the neck engaging portion 110 to the patient's head and neck region. The securing system 120 consists of fastener 122 that receives the end section 104 of the neck engaging portion 110 thus fastening the end section 104 with the end section 106 forming a neck engaging loop L1 around the patient's head and neck region. The opening 126 in the fastener 122 receives the end section 104 of the neck engaging portion 110. The first securing system 120 also provides a tightening mechanism to tighten and thereby secure the neck engaging portion 110 around the patient's head and neck/mouth region. As the end section 104 is passed through the opening 126, the length of the neck engaging portion 110 passing around the patient's head and neck region progressively reduces. Furthermore, as the length of the neck engaging portion 110 passing around the patient's head and neck region decreases, the neck engaging portion 110 contacting the patient's head and neck region gets tightened and tensioned around the patient's head and neck/mouth region. The securing system 120 also includes a locking mechanism as described above. The locking mechanism prevents any withdrawal of the end section 104 of the neck engaging portion 110 and thus prevents loosening or decrease in tensioning of the neck engaging portion 110 around the patient's head and neck region. Such a mechanism is particularly helpful for use by emergency personnel to secure the neck and head engaging portion 110 in position by rapidly, and extremely securely, tightening the neck and head engaging portion 110 around the patient's head and neck region. In the illustrated example the fastener 122 is placed just to the right (the patient's left) of the patient's mouth. The tube holder portion 150 is positioned relative to the fastener 122 so that when the fastener is placed in this position the tube holder portion 150 is positioned and oriented suitably for locating (and securing) a tube such as an endotracheal tube.

The tube holder portion 150 is used to secure the tube T by operation of the second securing arrangement 160. The second securing arrangement 160 includes the fastener 154 that is used to enable the elongate tube holder body 152 to secure the tube T. The fastener 154, positioned on end section 106 of the neck engaging portion 110, receives a section of the tube holder body 152 and fastens the tube holder body 152 to the neck and head engaging portion 110 forming a tube engaging loop L2. The tube engaging loop L2 passes around the tube T. The securing arrangement 160 also provides a tightening mechanism to tighten and thereby secure the tube holder body 152 around the tube T. As the tube holder body 152 is pulled by a user through the opening 126, the length of the tube holder body 152 passing around the tube T progressively reduces. Furthermore, as the length of the tube holder body 152 passing around the tube T decreases, the tube holder body 152 gets tightened and tensioned against the tube T. The securing system 160 also includes a locking mechanism as described above, in particular in relation to FIG. 5. The locking mechanism prevents the withdrawal of the tube holder body 152 from the opening 166 of the fastener 154 and thus prevents a loosening or decrease in tensioning of the tube holder body 152 around the tube T. Such a mechanism is particularly helpful for use by emergency personnel to secure the tube 1′ in the airway of the patient by rapidly, and securely, tightening the tube holder body 152 around the tube T.

The first securing arrangement 120 and the second securing arrangement 160 thus support and secure the tube T from movement and retain the tube T in position in the mouth and in the case of an endotracheal tube in the airway of the patient. The first securing arrangement 120 secures the neck engaging portion 110 thus securing the tube holder 10 relative to the patient. The tube holder portion 150 is connected to the neck engaging portion 110. Activation of the first securing arrangement 120 forms the neck engaging loop LI passing around the neck and secures the neck engaging portion 110 relative to the patient. Activation of the first securing arrangement 120 also positions and retains the tube holder portion 150 in the mouth region of the patient, in this embodiment, when the first securing arrangement 120 is positioned just to the side of the patient's mouth. The second securing arrangement 160 forms the tube engaging loop L2 and secures a previously free region of the tube holder body 152 to the neck engaging portion 110 and also secures the tube T to the tube holder portion 150. Advantageously, upon activation of the first securing arrangement 120 and the second securing arrangement 160, the neck engaging loop L1 is formed in a plane that is substantially perpendicular to the plane of the tube engaging loop L2. The mutually perpendicular planes of the neck engaging loop L1 and the tube engaging loop L2 facilitate correct positioning of the tube T, for example within the airway of the patient.

Tube holder 10 may he manufactured from commercially available materials such as but not limited to injection moulded plastic materials. Furthermore, the neck engaging portion 110 and the tube holder portion 150 may be manufactured as a unitary body by way of an injection moulding process (or possibly some alternative plastics moulding or forming process). Although considered advantageous, both for economy and convenience (since the illustrated embodiment requires no assembly prior to or during use) the present disclosure is not limited to manufacture of the neck engaging portion 110 and the tube holder portion 150 as a unitary body. Furthermore, the preferred embodiment presents components such as the first fastener 122 and the second fastener 154 being formed integrally with the neck engaging portion 110. However, the present disclosure is not limited to the integral formation or the positioning of the first fastener 122 and the second fastener 154.

The tube holder 10 is also not restricted for use with tubes of any particular size, since the tube holder portion 150 can be tightened about, and secure, tubes of a wide range of sizes. Similarly, a single tube holder 10 may be used effectively on patients of varying sizes, although it may be desirable to have ‘adult’ and ‘child’ sizes.

Of course, variations to the described and illustrated embodiment are possible. For example, the size and shape of the neck engaging portion 110 and the tube holder portion 150 may be varied without departing from the teachings disclosed herein. It should be understood by a person skilled in the art that the dimensions and materials specified in the non-limiting embodiments are for exemplary purposes only and other dimensions and materials may be used without departing from the scope of the invention.

It will be apparent to those skilled in the art that additional various modifications and variations can be made consistent with the present invention without departing from the scope or spirit of the invention. For example, various features within the several embodiments disclosed herein can be combined with features from other tube holder assembly embodiments. Other embodiments consistent with the invention will be apparent to those skilled in the art from consideration of the specification and practice of the invention disclosed herein. It is intended that the specification and examples be considered as exemplary only.

It is to be understood that, if any prior art publication is referred to herein, such reference does not constitute an admission that the publication forms a part of the common general knowledge in the art, in Australia or any other country.

In the claims which follow, and in the preceding description, except where the context requires otherwise due to express language or necessary implication, the word “comprise” and variations such as “comprises” or “comprising” are used in an inclusive sense, i.e. to specify the presence of the stated features but not to preclude the presence or addition of further features in various embodiments of the apparatus and method as disclosed herein.

1.-43. (canceled)

44. A medical tube holder, for securing a tube relative to a patient, comprising:
an elongate patient engaging portion for passing around at least a part of a neck and mouth region of a patient to secure the medical tube holder to the patient; and
a tube holding portion for passing around at least a part of a medical tube to secure the medical tube relative to the medical tube holder;
wherein, in use, the patient engaging portion and the tube holding portion retain and support the medical tube in position relative to the mouth of the patient; and
wherein, in use, when the tube-holding-portion is passed around the medical tube it defines a loop, and the loop is partially defined by a part of the patient engaging portion.

45. The medical tube holder of claim 44 wherein the patient-engaging-portion is integrally formed with the tube holding portion.

46. The medical tube holder of claim 44 further comprising:
a patient-engaging-portion securing arrangement for securing the patient engaging portion around at least a part of a neck and mouth region of a patient; and
a tube-holding-portion securing arrangement adapted for securing the tube to the tube holding portion and positioning the tube in the mouth of the patient;
wherein, in use, the patient-engaging-portion securing arrangement and the tube-holding-portion securing arrangement are adapted to retain and support the tube in position relative to the mouth of the patient.

47. The medical tube holder of claim 46 wherein the patient-engaging-portion securing arrangement further comprises:
a patient-engaging-portion tightening arrangement to facilitate tightening of the patient engaging portion around the patient's neck and mouth region; and
a patient-engaging-portion locking arrangement that prevents loosening of the patient engaging portion while allowing further tightening of the patient engaging portion around the neck and mouth region of the patient.

48. The medical tube holder of claim 46 wherein the tube-holding-portion securing arrangement further comprises:
a tube-holding-portion tightening arrangement to facilitate tightening of the tube holding portion around the tube; and
a tube-holding-portion locking arrangement that prevents loosening of the tube holding portion while allowing further tightening of the tube holding portion around the tube.

49. The medical tube holder of claim 46 wherein the patient-engaging-portion securing arrangement and/or the tube-holding-portion securing arrangement comprise at least one toothed ratchet portion, a pawl configured to engage the at least one toothed ratchet and a through hole for receiving the toothed ratchet portion therein.

50. The medical tube holder of claim 46 wherein the patient-engaging-portion securing arrangement is integrally formed with the patient engaging portion and wherein the tube-holding-portion securing arrangement is integrally formed with the tube holding portion.

51. The medical tube holder of claim 45 wherein the entire medical tube holder is integrally formed from a single piece of plastic.

52. The medical tube holder of claim 46 wherein the tube holding portion is arranged relative to the patient-engaging-portion securing arrangement so that tube holding portion can be appropriately positioned for securing a medical tube and retaining the medical tube in the mouth and an airway of the patient by positioning the patient-engaging-portion securing arrangement adjacent one side of the mouth of the patient.

53. The medical tube holder of claim 44, wherein the elongate patient engaging portion includes a positioning indicator thereon, and wherein the tube holding portion is arranged relative to the positioning indicator so that when the positioning indicator is positioned adjacent one side of the mouth of a patient the tube holding portion is appropriately positioned for securing a medical tube and retaining the medical tube in the mouth and an airway of the patient.

54. A method of securing a medical tube relative to a patient, the method comprising the steps of:
passing an elongate patient engaging portion of a medical tube holder according to any one of the preceding claims around at least a part of a neck and mouth region of a patient;
passing a tube holding portion of the medical tube holder around at least a part of a medical tube; and
securing the medical tube relative to the mouth of the patient.
